(19) United States
(12) Patent Application Publication
(10) Pub. No.: US 2023/0131516 A1
(43) Pub. Date: Apr. 27, 2023

(54) GAMMA-CAPROLACTONE PRECURSOR-AROMA COMPOUND AND PREPARATION METHOD AND USE THEREOF

(71) Applicant: CHINA TOBACCO SICHUAN INDUSTRIAL CO., LTD., Chengdu (CN)

(72) Inventors: Dikai ZHU, Chengdu (CN); Feiyan TAO, Chengdu (CN); Yu DING, Chengdu (CN); Liutianyi YANG, Chengdu (CN); Dandan CHANG, Chengdu (CN); Tao CUI, Chengdu (CN); Yiming WANG, Chengdu (CN); Xuejian ZHANG, Chengdu (CN); Juan YANG, Chengdu (CN); Qingguo YANG, Chengdu (CN); Lei WU, Chengdu (CN); Guangfu SONG, Chengdu (CN); Gang GUO, Chengdu (CN); Ning LI, Chengdu (CN); Pengcheng ZHU, Chengdu (CN)

(73) Assignee: CHINA TOBACCO SICHUAN INDUSTRIAL CO., LTD., Chengdu (CN)

(21) Appl. No.: 17/972,665

(22) Filed: Oct. 25, 2022

(30) Foreign Application Priority Data
Oct. 27, 2021 (CN) 202111256151.X

(51) Int. Cl. A24B 15/36; C07D 405/06; C11B 9/00

(52) U.S. Cl. CPC A24B 15/36; C07D 405/06; C11B 9/0096

(57) ABSTRACT

A γ-caprolactone precursor-aroma compound and a preparation method and uses thereof are disclosed. The preparation method specifically includes: dissolving γ-caprolactone in a sufficient amount of a solvent, adding an alkali at −78° C. to 0° C., stirring the resulting mixture to allow a reaction for 20 min or more, adding 2-acetylpyridine, further stirring at −78° C. to 0° C. to allow a reaction for 30 min or more, quenching the reaction, and finally subjecting the resulting reaction system to post-treatment, separation, and purification to obtain the target lactone precursor-aroma compound. The precursor-aroma compound of the present disclosure has stable properties in a normal temperature environment, can uniformly release an aroma under heating, increase and enrich the types of lactone fragrances, broaden an application range of lactone fragrances and acylpyridines, and overcome the defects of lactone and acylpyridine themselves, such as high volatility, small threshold, strong smell, and easy loss during processing.

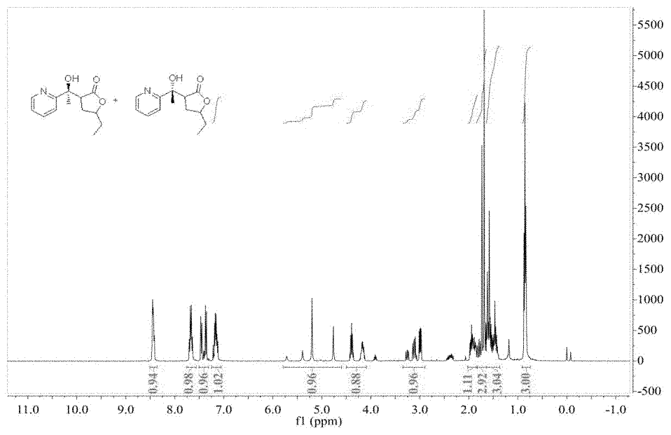

### CROSS REFERENCE TO THE RELATED APPLICATIONS

This application is based upon and claims priority to Chinese Patent Application No. 202111256151.X, filed on Oct. 27, 2021, the entire contents of which are incorporated herein by reference.

### TECHNICAL FIELD

The present disclosure relates to the technical field of tobacco essences and more particularly to a γ-caprolactone precursor-aroma compound and a preparation method and use thereof.

### BACKGROUND

γ-caprolactone (FEMA #2556, CAS #695-06-7) exhibits characteristics similar to coumarin but does not face the legal and regulatory risks of using coumarin. γ-caprolactone has sweet butterfat and flavors of lactose, tobaccos, coumarin, and coconuts. Thus, γ-caprolactone is often used to prepare bean-flavored and milk-flavored tobacco essences to improve the flavor of the smoke concentration and increase the sweet bean and milk flavor of tobacco. Pyridine compounds are a group of heterocyclic compounds that are the most widely developed and used in the field of tobacco essences. 2-acetylpyridine has the characteristic flavors of popcorn, nut, and tobacco and is often used to prepare baking-flavored and milk-flavored tobacco essences to enhance the nut, baking, and caramel sweetness flavors and improve the flavor of the smoke concentration.

However, the above two molecules both have the characteristics of high volatility and low threshold. Thus, they will bring about the disadvantages of easy volatilization and inconstant content when they are directly used in a tobacco formula, thereby affecting the quality and stability of the tobacco product.

A precursor-aroma compound, also known as an aroma precursor, refers to a precursor of an aroma, and the precursor is synthesized from one or more highly volatile or easily-sublimated aromas by physical or chemical means. The aroma precursor itself has no fragrance or has a fragrance that is not obvious and thus exhibits weak volatilization and stable chemical properties at room temperature. Therefore, the aroma precursor can be stored for a long time and can release desired flavor component only when a tobacco product is burned or heated so as to enhance the flavor of tobacco products. The flavor enhancement with an aroma precursor can reduce the loss of fragrance during processing and storage while providing a tobacco product with unique characteristics. The aroma precursor plays an important role in reducing the cost of fragrances used in tobacco products and improving the quality and stability of tobacco products.

### SUMMARY

An objective of the present disclosure is to provide a γ-caprolactone precursor-aroma compound and a preparation method and use thereof to solve the above problems. In the present disclosure, a novel precursor-aroma compound is prepared based on the covalent linking of γ-caprolactone and baking-flavor acylpyridine, which has high stability and strong ability to withstand processing and can overcome the disadvantages of γ-caprolactone and acylpyridine, such as high volatility, small threshold, strong smell, and easy loss during processing. The fragrance substances of γ-caprolactone and acylpyridine can be released at a high temperature (e.g., during smoking) to improve the smoking quality of a cigarette and enhance the flavor and aroma of the cigarette.

The present disclosure adopts the following technical solutions to achieve the above objective:

In a first aspect, the present disclosure provides a γ-caprolactone precursor-aroma compound with a structural formula as follows:

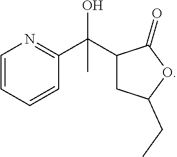 [SMILES: CCC1CC(C(C)(O)c2ccccn2)C(=O)O1]

In a second aspect, the present disclosure further provides a preparation method for the γ-caprolactone precursor-aroma compound, where a preparation reaction formula is as follows:

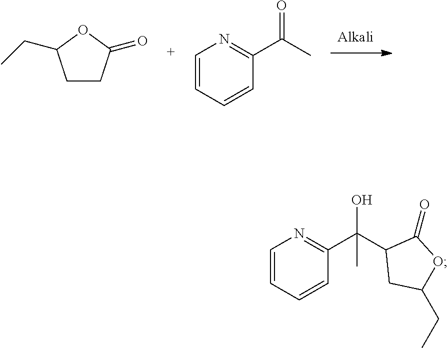

and

The preparation method specifically includes dissolving γ-caprolactone in a sufficient amount of a solvent, adding an alkali at −78° C. to 0° C., stirring the resulting mixture to allow a reaction for 20 min or more, adding 2-acetylpyridine, and further stirring at −78° C. to 0° C. to allow a reaction for 30 min or more, quenching the reaction, and finally subjecting the resulting reaction system to post-treatment, separation, and purification to obtain the target lactone precursor-aroma compound.

As a further solution, the solvent is one or more selected from the group consisting of diethyl ether, methyl tert-butyl ether (MTBE), tetrahydrofuran (THF), dioxane, methyltetrahydrofuran (MTHF), dichloromethane (DCM), 1,2-dichloroethane (1,2-DCE), dimethyl sulfoxide (DMSO), and petroleum ether.

As a further solution, the alkali is one or more selected from the group consisting of BuLi, LDA, LiHMDS, NaNH₂, NaH, NaOC(CH₃)₃, KOC(CH₃)₃, NaOEt, and KOEt.

As a further solution, the γ-caprolactone, the alkali, and the 2-acetylpyridine are in a molar ratio of 1:(4-6):(1-1.5).

As a further solution, the quenching of the reaction and the post-treatment include adding water to quench the reaction, separating the resulting organic phase out, washing with saturated brine, drying with anhydrous sodium sulfate, filtering, removing the solvent through vacuum distillation, and subjecting the residue to separation by silica gel column chromatography.

As a further solution, the reaction is conducted at −70° C. to 0° C. The first stirring is conducted for 20 min to 60 min, and the second stirring is conducted for 30 min to 12 h.

In a third aspect, the present disclosure also provides a use of the γ-caprolactone precursor-aroma compound or a γ-caprolactone precursor-aroma compound prepared by the preparation method as a fragrance, specifically including adding the γ-caprolactone precursor-aroma compound to a product that releases an aroma during combustion or heating at an amount 0.00001% to 10% of the weight of the product. For example, the γ-caprolactone precursor-aroma compound can be used in incense, candles, or fireplace fuels that produce a particular aroma; sauces, stew ingredients, or vegetable oil with a special flavor; or other substances such as tobaccos that release a flavor during combustion or heating.

In a fourth aspect, the present disclosure also provides a use of the γ-caprolactone precursor-aroma compound or a γ-caprolactone precursor-aroma compound prepared by the preparation method in tobacco, specifically including adding the γ-caprolactone precursor-aroma compound to the tobacco at an amount 0.00001% to 2% of the weight of the tobacco.

As a further solution, the γ-caprolactone precursor-aroma compound is added to the tobacco as follows: top dressing, casing, or sheet-adding or dissolving the γ-caprolactone precursor-aroma compound in water or alcohol or a mixture of the two and spraying or injecting the resulting solution on/into the tobacco. Tobacco is a mixed or flue-cured finished product or a component of a finished product formula.

The present disclosure has the following beneficial effects:

The precursor-aroma compound of the present disclosure has stable properties in a normal temperature environment, can uniformly release an aroma under heating, increase and enrich the types of lactone fragrances, broaden an application range of lactone fragrances and acylpyridine, and overcome the defects of lactone and acylpyridine themselves, such as high volatility, small threshold, strong smell, and easy loss during processing.

### BRIEF DESCRIPTION OF THE DRAWING

To describe the technical solutions of the embodiments of the present disclosure more clearly, the accompanying drawings required for describing the embodiments or the prior art are briefly described below. Apparently, the accompanying drawing in the following description merely shows some embodiments of the present disclosure, and persons of ordinary skill in the art may still derive other accompanying drawings from these accompanying drawings without creative efforts.

FIGURE shows a proton nuclear magnetic resonance (1H NMR) of the precursor-aroma compound LSD8 of the present disclosure.

### DETAILED DESCRIPTION OF THE EMBODIMENTS

To make the objectives, technical solutions, and advantages of the present disclosure clear, the technical solutions of the present disclosure will be described in detail below. Apparently, the described examples are merely a part, rather than all, of the examples of the present disclosure. All other embodiments obtained by persons of ordinary skill in the art based on the examples of the present disclosure without creative efforts should fall within the protection scope of the present disclosure.

### Example 1

25 mL (50 mmol) of lithium diisopropylamide was placed in a round-bottomed flask and stirred at −60° C. for 20 min. Subsequently, 1.1 g (10 mmol) of γ-caprolactone was dissolved in 10 mL of anhydrous THF. The resulting solution was slowly added dropwise to the lithium diisopropylamide solution, and the resulting mixture was stirred at −60° C. for 30 min to obtain the reaction system. 1.21 g (10 mmol) of 2-acetylpyridine was dissolved in 20 mL of anhydrous THF. The resulting solution was slowly added dropwise to the above reaction system. The resulting mixture was stirred at −60° C. for 40 min, and 30 mL of water was added for quenching. The resulting reaction system was concentrated to remove the THF, 100 mL of DCM was added for extraction, and the resulting organic phase was washed with saturated brine and dried with anhydrous sodium sulfate. The solvent was evaporated under reduced pressure to obtain a solid, and the solid was purified through column chromatography to obtain 1.65 g of a target compound (NZ1) with a yield of 70%.

Test results were shown in the FIGURE, where the structural characterization was as follows:

¹HNMR (400 MHz, CDCl₃): δ, ppm 0.80-0.87 (m, 3H), 1.37-1.64 (m, 3H), 1.65-1.82 (m, 3H), 1.84-2.00 (m, 1H), 2.97-3.28 (m, 1H), 4.07-4.49 (m, 1H), 4.60-5.68 (m, 1H), 7.13-7.19 (m, 1H), 7.31-7.50 (m, 1H), 7.56-7.75 (m, 1H), 8.42-8.46 (m, 1H).

### Example 2

25 mL (50 mmol) of lithium diisopropylamide was placed in a round-bottomed flask and stirred at −20° C. for 20 min. Subsequently, 1.1 g (10 mmol) of γ-caprolactone was dissolved in 10 mL of anhydrous THF. The resulting solution was slowly added dropwise to the lithium diisopropylamide solution, and the resulting mixture was stirred at −60° C. for 30 min to obtain the reaction system. 1.21 g (10 mmol) of 2-acetylpyridine was dissolved in 20 mL of anhydrous THF. The resulting solution was slowly added dropwise to the above reaction system, the resulting mixture was stirred at −60° C. for 40 min, and 30 mL of water was added for quenching. The resulting reaction system was concentrated to remove the THF, 100 mL of DCM was added for extraction, and the resulting organic phase was washed with saturated brine and dried with anhydrous sodium sulfate. The solvent was evaporated under reduced pressure to obtain a solid, and the solid was purified through column chromatography to obtain 1.04 g of a target compound (NZ1) with a yield of 44%.

### Example 3

30 mL (60 mmol) of lithium diisopropylamide was placed in a round-bottomed flask and stirred at −60° C. for 20 min. Subsequently, 1.1 g (10 mmol) of γ-caprolactone was dissolved in 10 mL of anhydrous THF. The resulting solution was slowly added dropwise to the lithium diisopropylamide solution, and the resulting mixture was stirred at −60° C. for 30 min to obtain the reaction system. 1.82 g (15 mmol) of 2-acetylpyridine was dissolved in 20 mL of anhydrous THF, and the resulting solution was slowly added dropwise to the above reaction system. The resulting mixture was stirred at −60° C. for 40 min, and 30 mL of water was added for quenching. The resulting reaction system was concentrated to remove the THF, 100 mL of DCM was added for extraction, and the resulting organic phase was washed with saturated brine and dried with anhydrous sodium sulfate. The solvent was evaporated under reduced pressure to obtain a solid, and the solid was purified through column chromatography to obtain 1.69 g of a target compound (NZ1) with a yield of 72%.

### Example 4

20 mL (40 mmol) of lithium diisopropylamide was placed in a round-bottomed flask and stirred at −60° C. for 20 min. Subsequently, 1.1 g (10 mmol) of γ-caprolactone was dissolved in 10 mL of anhydrous THF. The resulting solution was slowly added dropwise to the lithium diisopropylamide solution, and the resulting mixture was stirred at −60° C. for 30 min to obtain the reaction system. 1.21 g (10 mmol) of 2-acetylpyridine was dissolved in 20 mL of anhydrous THF, and the resulting solution was slowly added dropwise to the above reaction system. The resulting mixture was stirred at −60° C. for 40 min, and 30 mL of water was added for quenching. The resulting reaction system was concentrated to remove the THF, 100 mL of DCM was added for extraction, and the resulting organic phase was washed with saturated brine and dried with anhydrous sodium sulfate. The solvent was evaporated under reduced pressure to obtain a solid, and the solid was purified through column chromatography to obtain 1.37 g of a target compound (NZ1) with a yield of 58%.

### Example 5

25 mL (50 mmol) of lithium diisopropylamide was placed in a round-bottomed flask and stirred at −60° C. for 20 min. Subsequently, 1.1 g (10 mmol) of γ-caprolactone was dissolved in 10 mL of anhydrous THF, the resulting solution was slowly added dropwise to the lithium diisopropylamide solution, and the resulting mixture was stirred at −60° C. for 60 min to obtain the reaction system. 1.21 g (10 mmol) of 2-acetylpyridine was dissolved in 20 mL of anhydrous THF, and the resulting solution was slowly added dropwise to the above reaction system. The resulting mixture was stirred at −60° C. for 40 min, and 30 mL of water was added for quenching. The resulting reaction system was concentrated to remove the THF, 100 mL of DCM was added for extraction, and the resulting organic phase was washed with saturated brine and dried with anhydrous sodium sulfate. The solvent was evaporated under reduced pressure to obtain a solid, and the solid was purified through column chromatography to obtain 1.63 g of a target compound (NZ1) with a yield of 69%.

### Example 6

25 mL (50 mmol) of lithium diisopropylamide was placed in a round-bottomed flask and stirred at −60° C. for 20 min. Subsequently, 1.1 g (10 mmol) of γ-caprolactone was dissolved in 10 mL of anhydrous THF. The resulting solution was slowly added dropwise to the lithium diisopropylamide solution, and the resulting mixture was stirred at −60° C. for 30 min to obtain the reaction system. 1.21 g (10 mmol) of 2-acetylpyridine was dissolved in 20 mL of anhydrous THF, the resulting solution was slowly added dropwise to the above reaction system, the resulting mixture was stirred at −60° C. for 12 h, and 30 mL of water was added for quenching. The resulting reaction system was concentrated to remove the THF, 100 mL of DCM was added for extraction, and the resulting organic phase was washed with saturated brine and dried with anhydrous sodium sulfate. The solvent was evaporated under reduced pressure to obtain a solid, and the solid was purified through column chromatography to obtain 1.53 g of a target compound (NZ1) with a yield of 65%.

### Example 7

An NZ1 cigarette smoking evaluation experiment was now conducted to demonstrate that the compound of the present disclosure can improve the aroma of cigarette smoke. A specified amount of NZ1 was weighed and dissolved with ethanol. The resulting solution was added to a flue-cured tobacco shred at an amount of 0.003%, and the resulting mixture was rolled into an experimental cigarette. The same tobacco shred was taken, ethanol was added in the same proportion as above, and the resulting mixture was rolled into a blank cigarette. Comparative smoking was conducted, and results showed that, compared with the blank cigarette, the experimental cigarette had obvious sweetness, bean, and baking flavors, indicating an aroma enhancement effect.

In summary, the novel precursor-aroma compound of the present disclosure can uniformly release γ-caprolactone and acylpyridine to cigarette smoke when the cigarette is burned. The precursor-aroma compound has advantages such as a high boiling point, low volatility, and light smell and exhibits a prominent smoke flavoring effect when used for a cigarette. It can be seen that, when added to a cigarette, the precursor-aroma compound of the present disclosure can allow the cigarette to release a corresponding specific aroma and overcome the disadvantages of γ-caprolactone and acylpyridines themselves such as high volatility, small threshold, strong smell, and easy loss during processing.

The above are merely specific implementations of the present disclosure, and the protection scope of the present disclosure is not limited thereto. Any modification or replacement easily conceived by those skilled in the art within the technical scope of the present disclosure should fall within the protection scope of the present disclosure. Therefore, the protection scope of the present disclosure shall be subject to the protection scope of the claims. In addition, it should be noted that various specific technical features described in the above specific embodiments can be combined in any suitable manner, provided that there is no contradiction. To avoid unnecessary repetition, various possible combination modes of the present disclosure are not described separately. In addition, different implementations of the present disclosure can also be combined arbitrarily. The combinations should also be regarded as the content disclosed in the present disclosure, provided that they do not violate the ideas of the present disclosure.

What is claimed is:

1. A γ-caprolactone precursor-aroma compound with a structural formula as follows:

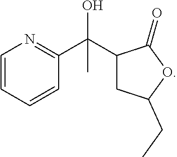 [SMILES: CCC1CC(C(C)(O)c2ccccn2)C(=O)O1]

2. A method of preparing the γ-caprolactone precursor-aroma compound according to claim 1, wherein a formula of a preparation reaction is as follows: 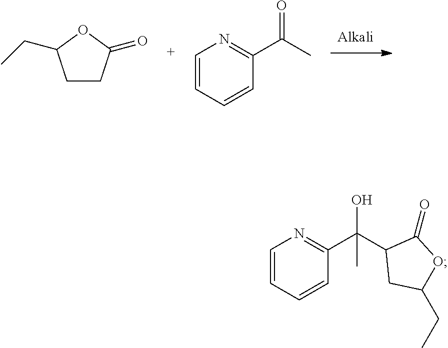
and
the method comprises: dissolving γ-caprolactone in a solvent, adding an alkali at −78° C. to 0° C. to form a first mixture, conducting a first stir to the first mixture for 20 min or more, adding 2-acetylpyridine to form a second mixture, conducting a second stir to the second mixture at −78° C. to 0° C. for 30 min or more, quenching the second mixture, and finally subjecting the second mixture to a post-treatment, a separation, and a purification to obtain the γ-caprolactone precursor-aroma compound.

3. The method of preparing the γ-caprolactone precursor-aroma compound according to claim 2, wherein the solvent is one or more selected from the group consisting of diethyl ether, methyl tert-butyl ether (MTBE), tetrahydrofuran (THF), dioxane, methyltetrahydrofuran (MTHF), dichloromethane (DCM), 1,2-dichloroethane (1,2-DCE), dimethyl sulfoxide (DMSO), and petroleum ether.

4. The method of preparing the γ-caprolactone precursor-aroma compound according to claim 2, wherein the alkali is one or more selected from the group consisting of BuLi, LDA, LiHMDS, NaNH₂, NaH, NaOC(CH₃)₃, KOC(CH₃)₃, NaOEt, and KOEt.

5. The method of preparing the γ-caprolactone precursor-aroma compound according to claim 2, wherein the γ-caprolactone, the alkali, and the 2-acetylpyridine are in a molar ratio of 1:(4-6):(1-1.5).

6. The method of preparing the γ-caprolactone precursor-aroma compound according to claim 2, wherein the quenching and the post-treatment comprise: adding a water to quench the second mixture, separating a resulting organic phase out, washing with a saturated brine, drying with anhydrous sodium sulfate, filtering, removing the solvent through a vacuum distillation, and subjecting a residue to the separation by a silica gel column chromatography.

7. The method of preparing the γ-caprolactone precursor-aroma compound according to claim 2, wherein the preparation reaction is conducted at −70° C. to 0° C.; the first stir is conducted for 20 min to 60 min; and the second stir is conducted for 30 min to 12 h.

8. A method of use of the γ-caprolactone precursor-aroma compound according to claim 1 as a fragrance, comprising: adding the γ-caprolactone precursor-aroma compound to a product with a release of an aroma during a combustion or a heating, wherein an amount of the γ-caprolactone precursor-aroma compound added to the product is 0.00001% to 10% of a weight of the product.

9. A method of use of the γ-caprolactone precursor-aroma compound according to claim 1 in a tobacco, comprising: adding the γ-caprolactone precursor-aroma compound to the tobacco, wherein an amount of the γ-caprolactone precursor-aroma compound added to the tobacco is 0.00001% to 2% of a weight of the tobacco.

10. The method of use of the γ-caprolactone precursor-aroma compound according to claim 9, wherein the γ-caprolactone precursor-aroma compound is added to the tobacco through top dressing, casing, or sheet-adding; or dissolving the γ-caprolactone precursor-aroma compound in a water or an alcohol or a mixture of the water and the alcohol to form a resulting solution, and spraying or injecting the resulting solution on/into the tobacco; and the tobacco is a mixed or flue-cured finished product or a component of a finished product formula.
